(19) United States
(12) Patent Application Publication
(10) Pub. No.: US 2005/0058674 A1
(43) Pub. Date: Mar. 17, 2005

(54) Moisturizing and lubricating compositions

(75) Inventors: Wael R. Joseph, Appleton, WI (US); Keisha Clarke, Appleton, WI (US); Duane G. Krzysik, Appleton, WI (US); Bernard J. Minerath III, Oshkosh, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/660,203

(22) Filed: Sep. 11, 2003

(51) Int. Cl. A61K 7/06; A61K 7/11; A61K 35/78

(52) U.S. Cl. 424/401; 424/744; 514/171; 514/565; 514/55

(57) ABSTRACT

The present invention is generally directed to moisturizing and lubricating compositions for use on the bodyfacing surface of an absorbent product or on one or both faces of a tissue product. The moisturizing and lubricating compositions comprise an emollient, a humectant, and immobilizing agent and a compatibilizing agent. Optionally, the moisturizing and lubricating compositions can comprise a dispersing agent.

### BACKGROUND OF THE INVENTION

This invention is directed to moisturizing and lubricating compositions which may be used on absorbent products such as disposable diapers, feminine napkins, tampons, interlabial pads and/or interlabial devices. The disclosed moisturizing and lubricating compositions may also be used in combination with tissue products such as bath and facial tissue. More particularly, this invention is directed towards moisturizing and lubricating compositions for use on the bodyfacing surface of an absorbent product, such as an interlabial pad, or on one or both surfaces of a tissue product, which improve the level of comfort to the wearer and may provide a skin health benefit.

The stratum corneum is the outer-most layer of the skin and is responsible for regulating skin water levels and functioning as a barrier against chemicals and other stressors found in the environment. The complex arrangement of lipids in the intercellular space of the stratum corneum is responsible for the establishment of normal barrier function. Multi-layered structures of cholesterol, ceramides, and fatty acids, as well as some other minor lipids, provide the major barrier to the transport of hydrophilic substances into or through the skin. The link between the barrier function and skin health is apparent from the skin inflammation caused by lipid extraction from the skin.

Skin barrier can be damaged due to a number of mechanisms. One mechanism for damage is physical abrasion, which may be caused by repeated rubbing of absorbent tissue products or disposable absorbent articles on the skin. With physical abrasion, layers of the skin are stripped away causing damage to the stratum corneum. Also, biological fluids, such as urine, feces, nasal and vaginal secretions, may contain a variety of components that can damage the stratum corneum. Some specific examples include proteases, lipases, bile acids, and fatty acids. Once the stratum corneum barrier is compromised, skin inflammation can occur.

Excessive hydration of the skin can also have a negative impact on skin barrier. The hydration level of diapered skin, for example, may reach between five and ten times that of undiapered skin. Frequent contact of diapered skin with fluids such as urine and feces may also contribute to increased hydration. Increased skin hydration disrupts skin lipid organization in the stratum corneum, and may increase the skin permeability of irritants, thus increasing the risk of skin inflammation.

Disposable absorbent articles such as diaper and incontinence garments as well as tissue products such as bath and facial tissue are commonly used to absorb body fluids and leave the skin dry. These products, in addition to absorbing and wiping fluids, however, also abrade the skin during use and frequently do not leave the skin completely dry and free of the body fluid after use. During frequent use of these products, the skin can become so dry and/or abraded as to appear red and be sore to the touch. To reduce this problem, additive formulations have been applied to absorbent articles and tissue products to provide lubricity and moisture. Once deposited on the skin, these products may provide a skin benefit by occluding the skin and protecting the stratum corneum until the damage is repaired.

To date, the formulations applied to absorbent articles and tissue products have not been completely satisfactory. Many formulations to date have proven to be unstable, even at slightly elevated temperatures and have tended to migrate into the product matrix prior to use where the formulation is only of minimal, if any benefit. Additionally, many formulations used to date have had very poor transfer rates from the product to the skin where it can be of use. As such, it is apparent that there is a commercial need for hydrophilic lubricating formulations suitable for use in combination with absorbent products such as disposable diapers and incontinence products, as well as tissue products such as facial tissue and bath tissue. It would be advantageous if the lubricating formulation could provide a moisturization benefit to alleviate skin dryness, as well as present a soft, aesthetically pleasing feel to reduce friction between the product and skin. Also, it would be advantageous if the lubricating formulations were formulated to be fluid during processing and rapidly solidify after application to the products.

### SUMMARY OF THE INVENTION

The present invention is generally directed to moisturizing and lubricating compositions for use in combination with absorbent products such as disposable diapers, incontinence garments, training pants, feminine pads, tampons, and interlabial pads as well as tissue products such as facial and bath tissue. The moisturizing and lubricating compositions, which are hydrophilic, are introduced onto the bodyfacing surface of the disposable article or onto one or both surfaces of a tissue product. Upon use, the lubricating composition contacts the skin and is at least partially transferred onto the skin to improve skin health.

In one embodiment described herein, at least a portion of the bodyfacing surface of a disposable absorbent article or at least one surface of a tissue product comprises a hydrophilic composition which is solid or semisolid at a temperature of about 30° C. to about 80° C. and comprises a humectant, an immobilizing agent, a compatibilizing agent, and an emollient. Optionally, the moisturizing and lubricating compositions of the present invention may comprise a skin barrier enhancing agent, such as sunflower oil or borage oil, and an antioxidant to stabilize the skin barrier enhancing agent. Additionally, a sterol or sterol derivative may be added to improve skin health.

The moisturizing and lubricating compositions of the present invention provide a reduction in the frictional discomfort and dryness associated with the use of absorbent articles and tissue products.

Briefly, therefore, the present invention is directed to a moisturizing and lubricating composition comprising from about 1% (by weight) to about 40% (by weight) of an emollient, from about 1% (by weight) to about 20% (by weight) of a humectant, from about 30% (by weight) to about 90% (by weight) an immobilizing agent, and from about 1% (by weight) to about 40% (by weight) of a compatibilizing agent. No more than about 50% (by weight) of the components of the moisturizing and lubricating composition are liquid at room temperature, and no less than about 50% of the components are solid at room temperature. Also, at least 85% (by weight) of the components of the moisturizing and lubricating composition form a single phase at a temperature of from about 45° C. to about 80° C.

The present invention is further directed to a moisturizing and lubricating composition comprising from about 1% (by weight) to about 40% (by weight) of a silicone, from about 1% (by weight) to about 20% (by weight) of a humectant, from about 30% (by weight) to about 90% (by weight) an immobilizing agent, from about 1% (by weight) to about 40% (by weight) of a compatibilizing agent and a dispersing agent. No more than about 50% (by weight) of the components of the moisturizing and lubricating composition are liquid at room temperature, and no less than about 50% of the components are solid at room temperature. Also, at least 85% (by weight) of the components of the moisturizing and lubricating composition form a single phase at a temperature of from about 45° C. to about 85° C.

### BRIEF DESCRIPTION OF THE DRAWINGS

FIG. 1 representatively shows a partially cut away top plan view of an absorbent article in a stretched and laid flat condition with the surface of the article which contacts the skin of the wearer facing the viewer.

FIG. 2 representatively shows a sectional view of the absorbent article of FIG. 1 taken along line 2-2.

FIG. 3 is a graph of the water vapor transmission rates of various moisturizing and lubricating compositions of the present invention as described in Example 1.

FIG. 4 is a graph of % Static hygroscopicity of various moisturizing and lubricating compositions of the present invention as described in Example 1.

### DETAILED DESCRIPTION OF THE PREFERRED EMBODIMENTS

In accordance with the present invention, it has been discovered that the bodyfacing surface of absorbent products or at least one surface of a tissue product can be treated with a moisturizing and lubricating composition to improve the overall performance of the absorbent or tissue product. The moisturizing and lubricating composition comprises a humectant, an immobilizing agent, a compatibilizing agent, and an emollient, and optionally a dispersing agent.

The moisturizing and lubricating compositions of the present invention are suitable for use on the bodyfacing surface of numerous absorbent products which contact the skin and/or vaginal/labial areas during use, as well as on various tissue products such as bath and facial tissue. Although the present invention is primarily described herein in relation to a disposable diaper, it should be recognized that the moisturizing and lubricating compositions are suitable for use on the bodyfacing surface of numerous other absorbent products such as feminine hygiene pads, pantiliners, micro-liners, interlabial pads, thong pads, tampons, adult incontinence garments, training pants and disposable swimming pants. Also, the moisturizing and lubricating compositions are suitable for use on one or both surfaces of tissue products such as facial tissue and bath tissue. Further, the moisturizing and lubricating compositions are suitable for use on wet wipes, dry wipes, and disposable towels.

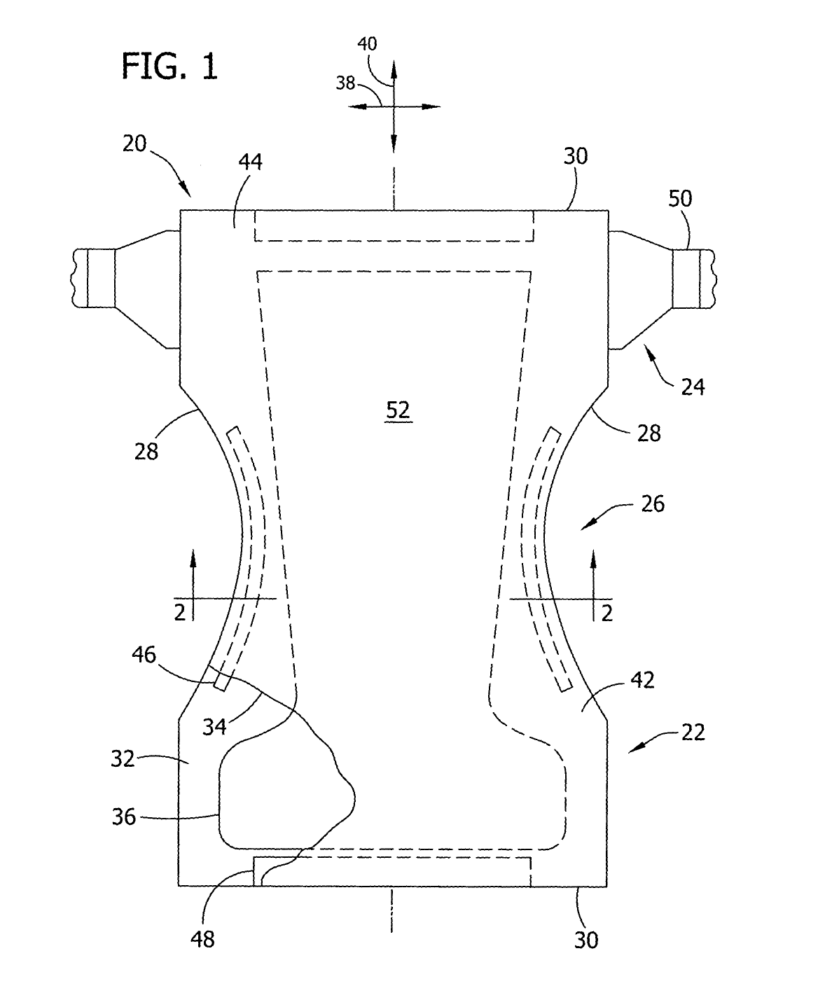

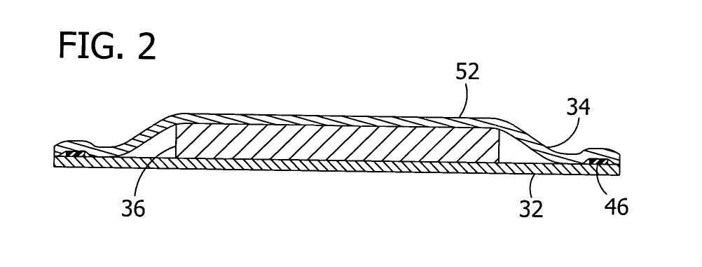

With reference to FIGS. 1 and 2, an integral absorbent garment article, such as a disposable diaper 20, generally defines a front waist section 22, a rear waist section 24, an intermediate section 26 which interconnects the front and rear waist section, a pair of laterally opposed side edges 28, and a pair of longitudinally opposed end edges 30. The front and rear waist sections include the general portions of the article which are constructed to extend substantially over the wearer's front and rear abdominal regions, respectively, during use. The intermediate section of the article includes the general portion of the article which is constructed to extend through the wearer's crotch region between the legs. The opposed side edges 28 define leg openings for the diaper and generally are curvilinear or contoured to more closely fit the legs of the wearer. The opposed end edges 30 define a waist opening for the diaper 20 and typically are straight but may also be curvilinear.

FIG. 1 is a representative plan view of the diaper 20 in a flat, non-contracted state. Portions of the structure are partially cut away to more clearly show the interior construction of the diaper 20, and the surface of the diaper which contacts the wearer is facing the viewer. The diaper 20 includes a substantially liquid impermeable outer cover 32, a porous, liquid permeable bodyside liner 34 positioned in facing relation with the outer cover 32, and an absorbent body 36, such as an absorbent pad, which is located between the outer cover and the bodyside liner. The diaper 20 also defines a lateral direction 38 and a longitudinal direction 40. Marginal portions of the diaper 20, such as marginal sections of the outer cover 32, may extend past the terminal edges of the absorbent body 36. In the illustrated embodiment, for example, the outer cover 32 extends outwardly beyond the terminal marginal edges of the absorbent body 36 to form side margins 42 and end margins 44 of the diaper 20. The bodyside liner 34 is generally coextensive with the outer cover 32, but may optionally cover an area which is larger or smaller than the area of the outer cover 32, as desired.

To provide improved fit and to help reduce leakage of body exudates from the diaper 20, the side margins 42 and end margins 44 of the diaper may be elasticized with suitable elastic members, such as leg elastic members 46 and waist elastic members 48. For example, the leg elastic members 46 may include single or multiple strands of elastic or elastomeric composites which are constructed to operably gather and shirr the side margins 42 of the diaper 20 to provide elasticized leg bands which can closely fit around the legs of the wearer to reduce leakage and provide improved comfort and appearance. Similarly, the waist elastic members 48 can be employed to elasticize the end margins 44 of the diaper 20 to provide elasticized waistbands. The waist elastics are configured to operably gather and shirr the waistband sections to provide a resilient, comfortably close fit around the waist of the wearer.

The elastic members 46 and 48 are secured to the diaper 20 in an elastically contractible condition so that in a normal under strain configuration, the elastic members effectively contract against the diaper 20. For example, the elastic members 46 and 48 may be elongated and secured to the diaper 20 while the diaper is in an uncontracted condition. In FIGS. 1 and 2, the elastic members 46 and 48 are illustrated in their uncontracted, stretched condition for the purpose of clarity. The diaper 20 may also include a pair of elasticized, longitudinally extending containment flaps (not shown), which are configured to maintain an upright, perpendicular arrangement in at least the intermediate section 26 of the diaper 20 to serve as an additional barrier to the lateral flow of body exudates. Suitable constructions and arrangements of containment flaps are well known to those skilled in the art.

Alternatively, the diaper 20 may include a pair of separate, elasticized and gathered leg gussets (not shown) or combination leg gussets/containment flaps (not shown) which are attached to the diaper along the side margins 42 in at least the intermediate section 26 of the diaper 20 to provide elasticized leg cuffs. Such gussets or combination gussets/containment flaps may be configured to extend beyond and bridge across the respective concave portion of the side margins 42.

The diaper 20, as representatively illustrated in FIGS. 1 and 2, may further include a pair of fasteners 50 employed to secure the diaper 20 about the waist of a wearer. Suitable fasteners 50 include hook-and-loop type fasteners, adhesive tape fasteners, buttons, pins, snaps, mushroom-and-loop fasteners, and the like. A cooperating side panel member can be associated with each fastener and may be constructed to be nonelasticized, or to be elastically stretchable at least along the lateral direction 38 of diaper 20.

The diaper may further include a surge management layer (not shown) positioned between the bodyside liner 34 and the absorbent body 36 which is configured to efficiently hold and distribute liquid exudates to the absorbent body 36. The surge management layer can prevent the liquid exudates from pooling and collecting on the portion of the diaper positioned against the wearer's skin, thereby reducing the level of skin hydration. Suitable constructions and arrangements of surge management layers are well known to those skilled in the art. Other suitable diaper components may also be incorporated on absorbent articles described herein.

The diaper 20 may be of various suitable shapes. For example, the diaper may have an overall rectangular shape, T-shape, or an approximately hour-glass shape. In the shown embodiment, the diaper 20 is I-shaped. Examples of diaper configurations suitable for use in connection with the instant application and other diaper components suitable for use on diaper are described in U.S. Pat. No. 4,798,603 issued Jan. 17, 1989 to Meyer et al.; U.S. Pat. No. 5,176,668 issued Jan. 5, 1993, to Bernardin; U.S. Pat. No. 5,176,672 issued Jan. 5, 1993 to Bruemmer et al.; U.S. Pat. No. 5,192,606 issued Mar. 9, 1993 to Proxmire et al.; and U.S. Pat. No. 5,509,915 issued Apr. 23, 1996 to Hanson et al., the disclosures of which are hereby incorporated by reference. The various aspects and configuration of the invention can provide distinctive combinations of softness, body conformity, reduced red-marking of the wearer's skin, reduced hydration, and improved containment of body exudates.

The various components of the diaper 20 are integrally assembled together employing various types of suitable attachment means, such as adhesive, sonic bonds, thermal bonds, or combinations thereof. In the shown embodiment, for example, the bodyside liner 34 and the outer cover 32 are assembled to each other and to the absorbent body 36 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Similarly, other diaper components, such as the elastic members 46 and 48 and the fasteners 50, may be assembled into the diaper 20 by employing the above-identified attachment mechanisms.

The outer cover 32 of the diaper 20, as representatively illustrated in FIGS. 1 and 2, may suitably be composed of material which is either liquid permeable or liquid impermeable. It is generally preferred that the outer cover 32 be formed from a material which is substantially impermeable to liquids. For example, a typical outer cover can be manufactured from a thin plastic film or other flexible liquid-impermeable material. For example, the outer cover 32 may be formed from a polyethylene film having a thickness of from about 0.012 millimeter (0.5 mil) to about 0.051 millimeter (2.0 mils). If it is desired to present the outer cover with a more clothlike feeling, the outer cover 32 may comprise a polyolefin film having a nonwoven web laminated to the outer surface thereof, such as a spunbond web of polyolefin fibers. For example, a stretch-thinned polypropylene film having a thickness of about 0.015 millimeter (0.6 mils) may have thermally laminated thereto a spunbond web of polypropylene fibers, which fibers have a thickness of about 1.5 to 2.5 denier per filament, which nonwoven web has a basis weight of about 17 grams per square meter (0.5 ounce per square yard). Methods of forming such clothlike outer covers are known to those skilled in the art. Further, the outer cover 32 may be formed of a woven or nonwoven fibrous web layer which has been totally or partially constructed or treated to impart a desired level of liquid impermeability to selected regions that are adjacent or proximate to the absorbent body 36.

Desirably, the outer cover 32 may be composed of a “breathable” material which permits vapors to escape from the absorbent body 36 while still preventing liquid exudates from passing through the outer cover 32. For example, the outer cover 20 is desirably constructed to be permeable to at least water vapor and has a water vapor transmission rate of at least about 1000 g/m²/24 hours, desirably at least about 1500 g/m²/24 hours, more desirably at least about 2000 g/m²/24 hours, and even more desirably at least about 3000 g/m²/24 hours. Materials which have a water vapor transmission rate less than those above do not allow a sufficient amount of air exchange and undesirably result in increased levels of skin hydration. As used herein, the phrase “water vapor transmission rate” (WVTR) refers to the WVTR value according to the Water Vapor Transmission Rate Test which is described in further detail herein below.

In a particular embodiment, the outer cover 20 is provided by a microporous film/nonwoven laminate material comprising a spunbond nonwoven material laminated to a microporous film. For example, the laminate may include a 0.6 osy (20.4 gsm) polypropylene spunbond material thermally attached to a 18.7 gsm stretched microporous film. The film may include from about 20 percent to about 75 percent by weight calcium carbonate particulates and the remainder primarily low density polyethylene. The film is then stretched which causes the polyethylene component to stretch while the particulates remain unstretched, thus causing voids to develop around the calcium carbonate particles in the film. The resulting laminate may define a water vapor transmission rate of from about 1000 to about 5000 g/m²/24 hours.

Examples of suitable breathable materials for the outer cover 20 are also described in U.S. Pat. No. 5,879,341 issued Mar. 9, 1999 to Odorzynski et al. and entitled “ABSORBENT ARTICLE HAVING A BREATHABILITY GRADIENT”; U.S. Pat. No. 5,843,056 issued Dec. 1, 1988, to Good et al. and entitled “ABSORBENT ARTICLE HAVING A COMOSITE BREATHABLE OUTER COVER”; and U.S. Pat. No. 5,855,999 issued Jan. 5, 1999 to McCormack et al. and entitled “BREATHABLE, CLOTH-LIKE FILM/NONWOVEN COMPOSITE”, the disclosures of which are herein incorporated by reference.

The absorbent body 36 of the diaper 20, as representatively illustrated in FIGS. 1 and 2, may suitably comprise a matrix of hydrophilic fibers, such as a web of cellulosic fluff, mixed with particles of a high-absorbency material commonly known as superabsorbent material. In a particular embodiment, the absorbent body 36 comprises a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be non-uniformly mixed. The fluff and superabsorbent particles may also be selectively placed into desired zones of the absorbent body 36 to better contain and absorb body exudates. The concentration of the superabsorbent particles may also vary through the thickness of the absorbent body 36. Alternatively, the absorbent body 36 may comprise a laminate of fibrous webs and superabsorbent material or other suitable means of maintaining a superabsorbent material in a localized area.

The absorbent body 36 may have any of a number of shapes. For example, the absorbent core may be rectangular, I-shaped, or T-shaped. It is generally preferred that the absorbent body 36 be narrower in the crotch area than in the front or rear portions of the diaper 20. The size and the absorbent capacity of the absorbent body 36 should be compatible with the size of the intended wearer and the liquid loading imparted by the intended use of the absorbent article.

The high-absorbency material can be selected from natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va., and DOW DRYTECH 2035LD polymer available from Dow Chemical Company located in Midland, Mich.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. As a general rule, the high absorbency material is present in the absorbent body in an amount of from about 5 to about 90 weight percent based on a total weight of the absorbent body 36.

Optionally, a substantially hydrophilic tissue wrapsheet (not shown) may be employed to help maintain the integrity of the airlaid fibrous structure of the absorbent body 36. The tissue wrapsheet is typically placed about the absorbent body over at least the two major facing surfaces thereof and composed of an absorbent cellulosic material, such as creped wadding or a high wet-strength tissue. In one aspect of the invention, the tissue wrapsheet can be configured to provide a wicking layer, which helps to rapidly distribute liquid over the mass of absorbent fibers comprising the absorbent body. In another aspect of the invention, the wrapsheet material on one side of the absorbent fibrous mass may be bonded to the wrapsheet located on the opposite side of the fibrous mass.

The bodyside liner 34, as representatively illustrated in FIGS. 1 and 2, suitably presents a bodyfacing surface which is compliant, soft feeling, and non-irritating to the wearer's skin. Further, the bodyside liner 34 may be less hydrophilic than the absorbent body 36, to present a relatively dry surface to the wearer, and may be sufficiently porous to be liquid permeable, permitting liquid to readily penetrate through its thickness. A suitable bodyside liner 34 may be manufactured from a wide selection of web materials, such as porous foams, reticulated foams, apertured plastic films, natural fibers (i.e., wood or cotton fibers), synthetic fibers (i.e., polyester or polypropylene fibers), or a combination of natural and synthetic fibers. The bodyside liner 34 is suitably employed to help isolate the wearer's skin from liquids held in the absorbent body 36.

Various woven and nonwoven fabrics can be used for the bodyside liner 34. For example, the bodyside liner 34 may be composed of a meltblown or spunbonded web of polyolefin fibers. The bodyside liner 34 may also be a bonded-carded web composed of natural and/or synthetic fibers. The bodyside liner 34 may be composed of a substantially hydrophobic material, and the hydrophobic material may, optionally, be treated with a surfactant, a wetting agent, or otherwise processed to impart a desired level of wettability and hydrophilicity.

In a particular embodiment, the bodyside liner 34 comprises a nonwoven, spunbond, polypropylene fabric composed of about 2.8-3.2 denier fibers formed into a web having a basis weight of about 20 grams per square meter and a density of about 0.13 gram per cubic centimeter. The fabric may be surface treated with about 0.3 weight percent of a surfactant mixture, which contains a mixture of AHCOVEL Base N-62 and GLUCOPOAN 220UP surfactant in a 3:1 ratio based on a total weight of the surfactant mixture. The ANCOVEL Base N-62 is purchased from Hodgson Textile Chemicals Inc., (Mount Holly, N.C.) and includes a blend of hydrogenated ethoxylated castor oil and sorbitan monooleate in a 55:45 weight ratio. The GLUCOPAN 220UP is purchased from Henkel Corporation and includes alkyl polyglycoside. The surfactant may be applied by any conventional means, such as spraying, printing, brush coating, or the like. The surfactant may be applied to the entire bodyside liner 34, or may be selectively applied to particular sections of the bodyside liner 34, such as the medial section along the longitudinal centerline of the diaper, to provide greater wettability of such sections.

The bodyside liner 34 of the absorbent article includes a moisturizing and lubricating composition on the bodyfacing surface 52 thereof. As noted above, the moisturizing and lubricating compositions of the present invention for use in combination with an absorbent article or tissue product comprise a humectant, an immobilizing agent, a compatibilizing agent, and emollient, and optionally a dispersing agent.

The moisturizing and lubricating compositions of the present invention are preferably hydrophilic in nature; that is, the compositions are attracted to, and retain, water. It has been discovered that hydrophilic moisturizing and lubricating compositions for use on the bodyfacing surface of absorbent products or on one or both surfaces of a tissue product typically introduce a higher level of moisture onto the skin or mucosal area, and retain the moisture in that area, as compared to hydrophobic compositions. As such, in accordance with the present invention, it is preferred that the moisturizing and lubricating compositions of the present invention be hydrophilic in nature.

One measurement of a composition's hydrophilicity is the composition's ability to dissolve or disperse in water. Typically, such dissolution/dispersion evaluations are done with heated water to improve the rate of dissolution/dispersion. The more soluble a composition is in water (typically compositions may be tested with water at a temperature of about 80° C. as the formulations may be solid at room temperature) the more hydrophilic the composition. As such, it is preferred that the moisturizing and lubricating compositions of the present invention have a high degree of solubility/dispersibility in heated water.

More specifically, the lubricating and moisturizing compositions of the present invention are preferably at least about 40%, more preferably at least about 45%, more preferably at least about 60%, more preferably at least about 70%, and still more preferably at least about 80% soluble/dispersible in deionized water at a temperature of about 80° C. A preferred moisturizing and lubricating composition of the present invention is about 80% soluble/dispersible in deionized water at a temperature of about 80° C. At these levels of solubility/dispersibility in 80° C. deionized water, the moisturizing and lubricating compositions of the present invention are sufficiently hydrophilic to provide the intended benefits.

The lubricating and moisturizing compositions of the present invention desirably have a melting point of from about 30° C. to about 80° C., more desirably from about 40° C. to about 70° C., and still more desirably from about 55° C. to about 75° C. With melting points in these ranges, the moisturizing and lubricating compositions of the present invention have a reduced tendency to flow easily and migrate into the interior of the product. This is important for at least two reasons. First, it is preferred that the majority of the moisturizing and lubricating composition remain on the surface of the product to allow direct interaction with the skin or mucous membrane it contacts. As such, a sufficiently high freezing temperature is desired such that the moisturizing and lubricating composition freezes, or solidifies, onto the substrate to which it is applied in a short amount of time to reduce the potential for run-off or migration. Therefore, it is preferred that the moisturizing and lubricating composition freeze onto the substrate in no more than about 3 seconds, preferably no more than about 0.25 seconds. With freezing rates in these ranges, the moisturizing and lubricating compositions of the present invention solidify quickly onto the substrate and the potential for migration is minimized.

Secondly, the melting point needs to be sufficiently high to provide sufficient stability for the composition. Stated another way, the melting points are desirably higher than the temperatures that the product to which the moisturizing and lubrication has been applied is exposed to during storage and transport, which may be as high as about 55° C.

Additionally, the moisturizing and lubricating compositions of the present invention desirably have a penetration hardness (needle penetration in millimeters according to ASTM D 1321, “Needle Penetration of Petroleum Waxes”) of from about 1 millimeter to about 200 millimeters, desirably from about 1 millimeter to about 120 millimeters, more desirably from about 1 millimeter to about 20 millimeters, and even more desirably from about 3 to about 17 millimeters. Penetration hardness of the moisturizing and lubricating compositions of the present invention may be important for two reasons. First, the softer the formulation (i.e., the higher the penetration hardness number) the more mobile the formulation will be, making the formulation more likely to migrate into the inner layers of the absorbent product. As such, it is typically desirable to have a penetration hardness of not more than 50 millimeters, desirably not more than about 20 millimeters to reduce this likelihood.

Second, very soft formulations tend to be more greasy/oily to the touch, which is typically not desirable on most absorbent and tissue products. By requiring the moisturizing and lubricating compositions of the present invention to have a penetration hardness as described above, these compositions are less likely to migrate yet maintain a silky, creamy feeling on the surface of an absorbent product.

The moisturizing and lubricating compositions of the present invention are preferably substantially non-irritating to the skin or mucous membrane to which they contact during use by the wearer; that is, it is preferred that the moisturizing and lubricating compositions not induce redness and/or swelling of the skin tissues when contacted with the skin of the wearer. Further, it is preferred that the moisturizing and lubricating compositions not interfere with or hinder the natural ability of the skin and mucous membranes to repair themselves from injury due to, for example, wiping or other abrasive activity. As discussed below, some of the moisturizing and lubricating compositions of the present invention may contain a fat or oil and/or a sterol or sterol derivative to facilitate repair of the skin from such damage.

The moisturizing and lubricating compositions of the present invention comprise an emollient, a humectant, an immobilizing agent, a compatibilizing agent, and optionally a dispersing agent. Other optional components may also be included in the moisturizing and lubricating compositions described herein.

The moisturizing and lubricating compositions of the present invention comprise from about 1% (by weight) to about 40% (by weight) of an emollient. As used herein, the term “by weight” refers to the total weight of the moisturizing and lubricating composition. Thus, if a moisturizing and lubricating composition is 25% (by weight) emollients and has a total weight of 100 grams, the compositions comprises 25 grams of emollient. As used herein, an emollient refers to a compound that smoothes, softens, soothes, supples, coats, lubricates, moisturizes, protects and/or cleanses the skin and/or mucous membranes, such as labial walls, upon contact.

Emollients suitable for use in the moisturizing and lubricating compositions of the present invention include, but are not limited to, petroleum based emollients, fatty acids, fatty acids esters, vegetable oils, hydrogenated vegetable oils, alkyl ethoxylates, fatty alcohols and silicones such as dimethicone, dimethiconol, PEG dimethicone, alkyl silicones, phenyl silicones, and silicone phospholipids, and combinations thereof. Preferred emollients include silicone based emollients such as Dow Corning 200 Fluids and Dow Corning 1503 Fluids.

Suitable petroleum based emollients include those hydrocarbons, or mixtures of hydrocarbons, having chain lengths of from 16 to 32 carbon atoms. Petroleum based hydrocarbons having these chain lengths include mineral oil (also known as “liquid petrolatum”) and petrolatum (also known as “mineral wax,” “petroleum jelly” and “mineral jelly”). Mineral oil usually refers to less viscous mixtures of hydrocarbons having from 16 to 20 carbon atoms. Petrolatum usually refers to more viscous mixtures of hydrocarbons having from 16 to 32 carbon atoms.

Suitable fatty acid ester emollients include those derived from C₁₂-C₂₈ fatty acids, preferably C₁₆-C₂₂ saturated fatty acids, and short chain, such as C₁-C₈, preferably C₁-C₃, monohydric alcohols. Examples include methyl palmitate, methyl stearate, isopropyl laurate, isopropyl myristate, butyl myristate, butyl stearate, octyl palmitate, isopropyl isostearate, isopropyl palmitate, ethylhexyl palmitate, and mixtures thereof. Suitable fatty acid ester emollients can also be derived from monoesters and diesters of both short chain, such as C₁-C₁₀, and longer chain fatty alcohols, such as C₁₂-C₂₈, preferably C₁₂-C₁₆, and shorter chain organic acids such as lactic acid, lauryl lactate and cetyl lactate. Additional examples include diisopropyl sebacate, dimethyl sebacate, dioctyl sebacate, dibutyl sebacate, diisopropyl adipate, and dicapryl adipate. In addition, mixtures of petroleum based emollients and fatty acid ester emollients can provide emollient systems that have a superior feel compared to the pure components individually.

Suitable alkyl ethoxylate type emollients include C₁₂-C₂₂ fatty alcohol ethoxylates having an average degree of ethoxylation of from about 2 to about 30. Preferably, the fatty alcohol ethoxylate emollient is selected from the group of lauryl, cetyl, and stearyl ethoxylates, and mixtures thereof, having an average degree of ethoxylation ranging from about 2 to about 23. Representative examples of such alkyl ethoxylates include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and ceteareth-10 (a mixture of cetyl and stearyl ethoxylates having an average degree of ethoxylation of 10). Additionally, alkyl ethoxylates with an HLB of from about 7 to about 14 are also useful as emulsifiers or compatibilizers/solubilizers of other emollients in the composition.

Suitable fatty acid-type emollients include acids having a carbon chain length of C₁₄-C₃₀ including myristic acid, palmitic acid, stearic acid, behenic acid, and mixtures thereof. Additionally, C₉-C₁₅ acids including caprylic acid, lauric acid, and the like are suitable fatty acid-type emollients.

Suitable fatty alcohol-type emollients include alcohols having a carbon chain length of C₁₄-C₃₀, including cetyl alcohol, stearyl alcohol, arachidyl alcohol, and behenyl alcohol and mixtures thereof. Additionally, C₉-C₁₅ alcohols including caprylic alcohol, cetearyl alcohol, coconut alcohol, decyl alcohol, hydrogenated tallow alcohol, lanolin alcohol, lauryl alcohol, myristyl alcohol, oleyl alcohol, palm alcohol, palm kernel alcohol, tallow alcohol, tridcyl alcohol and the like are suitable fatty alcohol-type emollients.

Another suitable type of emollient is a silicone, such as a polysilioxane compound. Generally, suitable polysiloxane materials for use include those having monomeric siloxane units having the following structure: 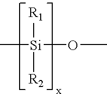
wherein x is a whole number from 1 to about 1,000,000 and R₁ and R₂ for each independent siloxane monomeric unit can each independently be hydrogen or any alkyl, aryl, alkenyl, alkaryl, arakyl, cycloalkyl, halogenated hydrocarbon, or other radical. Any of such radicals can be substituted or unsubstituted. R₁ and R₂ radicals of any particular monomeric unit may differ from the corresponding functionalities of the next adjoining monomeric unit. Additionally, the polysiloxane can be either a straight chain, a branched chain or have a cyclic structure. The radicals R₁ and R₂ can additionally independently be other silaceous functionalities such as, but not limited to siloxanes, polysiloxanes, silanes, and polysilanes. The radicals R₁ and R₂ may contain a variety of organic functionalities including, for example, alcohol, carboxylic acid, phenyl, and amine functionalities.

Exemplary radicals are methyl, ethyl, propyl, butyl, pentyl, hexyl, octyl, decyl, octadecyl, and the like. Exemplary alkenyl radicals are vinyl, allyl, and the like. Exemplary aryl radicals are phenyl, diphenyl, naphthyl, and the like. Exemplary alkaryl radicals are toyl, xylyl, ethylphenyl, and the like. Exemplary aralkyl radicals are benzyl, alphaphenylethyl, beta-phenylethyl, alpha-phenylbutyl, and the like. Exemplary cycloalkyl radicals are cyclobutyl, cyclopentyl, cyclohexyl, and the like. Exemplary halogenated hydrocarbon radicals are chloromethyl, bromoethyl, tetrafluorethyl, fluorethyl, trifluorethyl, trifluorotoyl, hexafluoroxylyl, and the like.

The viscosity of the useful polysiloxanes may vary widely. So long as the polysiloxane is flowable or can be made to be flowable for application, the polysiloxane viscosity is acceptable. This includes, but is not limited to, viscosity as low as 5 centistokes (at 37° C. as measured by a glass viscometer) to about 20,000,000 centistokes (at 37° C. as measured by a glass viscometer). A preferred range is from about 5 centistokes to about 5,000 centistokes. In one embodiment, a mixture of a low viscosity dimethicone (about 5 centistokes to about 350 centistokes) and a high viscosity dimethicone or dimethiconol or silicone gum (1,000,000 centistokes to about 20,000,000 centistokes) is preferred as the mixture produces a pleasant feeling material and provides additional lubricity. Preferred ranges of the low viscosity polysiloxane to high viscosity polysiloxane is from about 1:1 to about 10:1.

Preferred polysiloxane compounds for use as emollients in the present invention are disclosed in U.S. Pat. No. 5,059,282 (Ampulski, et al.). Particularly preferred polysiloxane compounds for use as emollients in the moisturizing and lubricating compositions of the present invention include phenyl-functional polymethylsiloxane compounds (e.g., Dow Corning 556 Cosmetic-Grade Fluid) and cetyl or stearyl functionalized dimethicones such as Dow 2502, General Electric SF1632 and Dow 2503 polysiloxane fluids. In addition to such substitution with phenyl-functional or alkyl groups, effective substitution may be made with amino, carboxyl, hydroxyl, ether, polyether, aldehyde, ketone, amide, ester, and thiol groups. Phenyl, amino, alkyl, carboxyl, and hydroxyl groups are preferred, with phenyl functional groups being most preferred.

The humectant component of the moisturizing and lubricating compositions of the present invention are generally present in an amount of from about 1% (by weight) to about 20% (by weight), preferably from about 5% (by weight) to about 15% (by weight). Humectants are typically cosmetic ingredients used to increase the water content of the top layers of the skin or mucous membrane, by helping control the moisture exchange between the product, the skin, and the atmosphere. Humectants may include primarily hydroscopic materials. Suitable humectants for inclusion in the moisturizing and lubricating compositions of the present invention include urocanic acid, N-Acetyl ethanolamine, aloe vera gel, arginine PCA, chitosan PCA, copper PCA, Corn glycerides, dimethyl imidazolidinone, fructose, glucamine, glucose, glucose glutamate, glucuronic acid, glutamic acid, glycereth-7, glycereth-12, glycereth-20, glycereth-26, glycerin, honey, hydrogenated honey, hydrogenated starch hydrolysates, hydrolyzed corn starch, lactamide MEA, lactic acid, lactose lysine PCA, mannitol, methyl gluceth-10, methyl gluceth-20, PCA, PEG-2 lactamide, PEG-10 propylene glycol, polyamino acids, polysaccharides, polyamino sugar condensate, potassium PCA, propylene glycol, propylene glycol citrate, saccharide hydrolysate, saccharide isomerate, sodium aspartate, sodium lactate, sodium PCA, sorbitol, TEA-lactate, TEA-PCA, Urea, Xylitol, and the like and mixtures thereof. Preferred humectants include polyols, glycerine, ethoxylated glycerine, polyethylene glycols, hydrogenated starch hydrolsates, propylene glycol, silicone glycol and pyrrolidone carboxylic acid.

The immobilizing agent component of the moisturizing and lubricating compositions of the present invention are generally present in an amount of from about 30% (by weight) to about 90% (by weight), preferably from about 40% (by weight) to about 70% (by weight). The immobilizing agent will reduce the tendency of the emollient and humectant to migrate or flow by keeping the emollient and humectant primarily localized. In addition to immobilizing the emollient and humectant, the immobilizing agent may provide a slight tackiness to the moisturizing and lubricating composition, which may improve transfer of the composition to skin or membrane of the wearer.

Suitable immobilizing agents for use with the moisturizing and lubricating compositions of the present invention include metal soaps including aluminum, calcium, magnesium, and zinc stearates, C₁₄-C₂₂ fatty alcohols, C₁₂-C₂₂ fatty acids, solid fatty acid esters, C₁₂-C₂₂ fatty alcohol ethoxylates having an average degree of ethoxylation ranging from about 2 to about 30, and high molecular weight (greater than about 720) polyethylene glycols (a polymer of ethylene oxides) that are solids at room temperature having the following empirical formula:
H(OCH₂CH₂)ₓOH
wherein x is the degree of ethoxylation and is an average value of at least about 20 moles or greater. Preferably, x is an average value of from about 20 to about 1,000, and even more preferably from about 100 to about 500. Particularly preferred high molecular weight polyethylene glycols are polyethylene glycols having the technical names of PEG 1000 (where x=20), PEG 3350 (where x=75), PEG 6000 (where x=125), PEG 8000 (where x=150), and PEG 10,000 (where x=220).

Preferred fatty alcohol immobilizing agents include C₁₆-C₁₈ fatty alcohols and fatty acids such as myristyl, cetyl, cetearyl, stearyl, behenyl alcohols and acids, and mixtures thereof. The preferred immobilizing agents increase the rate of crystallization of the emollient causing the emollient to crystallize rapidly.

Along with the emollient, humectant, and immobilizing agent, the moisturizing and lubricating compositions described herein comprise a compatibilizing agent. Compatibility of the overall moisturizing and lubricating composition is important for processability and stability. Incompatible compositions require a more rigorous process to ensure that mixing is complete so as to prevent the separation of the different components in the composition. More mixing requires higher energy consumption, which leads to an increase in the cost of manufacturing the products. Further, it may be very difficult for an incompatible composition to maintain acceptable stability during the life of the product, starting with shipping, transportation, and storage prior to ultimate use by the consumer. Many incompatible ingredients may tend to slowly separate from the surface of the product to which they are applied resulting in a loss of the properties of the overall composition and a potential loss in the intended benefits.

Some components described herein as components of the moisturizing and lubricating compositions of the present invention may be incompatible with the preferred humectant, glycerin. Specifically, it has been discovered that several immobilizing agents, including high molecular weight polyethylene glycols, are actually incompatible with glycerin. As such, in order to ensure a high degree of compatibility and a substantially homogeneous moisturizing and lubricating composition, the compositions described herein include a compatibilizing agent. For example, the compatibilizing agent is capable of compatibilizing glycerin and high molecular weight polyethylene glycols. The compatibilizing agent may be selected from propylene glycol, butylene glycol, 1,3 butylene glycol, low molecular weight polyethylene glycols (molecular weights of less than about 720 and liquid at room temperature such as, for example, PEG 600), methoxyisopropanol, dipropylene glycol propyl ether, dipropylene glycol butyl ether, dipropylene glycol, methyl propanediol, and soluble/dispersible polypropylene glycols. The compatibilizing agent is present in the moisturizing and lubricating compositions of the present invention in an amount of from about 1% (by weight) to about 40% (by weight).

As mentioned above, the moisturizing and lubricating compositions described herein may optionally comprise a dispersing agent. Because some silicones, which may be introduced into the compositions as emollients as discussed above, may be incompatible with glycerin and some glycols, a dispersing agent may be added to improve the compatibility of silicones when they are introduced into the composition. Useful dispersing agents include polyether ethoxylated/propoxylated modified polydimethylsiloxanes which are fully or partially compatible with polydimethylsiloxanes, silicone polyethers having at least 30% siloxane, between 10% and 40% ethoxylation and between 0% and 40% propoxylation. For example, Dow Corning 5329 may be introduced as a dispersing agent to obtain the desired benefits. The weight ratio of silicone dispersing agent to silicone is preferably 3:1, more desirably 2:1, and even more desirably 1:1.

Although the liquid components of the moisturizing and lubricating compositions described herein are important as they provide plasticity and help to avoid a product that is too hard, brittle or flaky and thus uncomfortable, moisturizing and lubricating compositions that contain a high proportion of components that are liquid at room temperature are more difficult to process. Upon freezing, solid components, especially immobilizing agents, are important for providing a network that is capable of supporting the liquid components within it and, therefore, preventing their migration through the substrate. When the solid portion of the moisturizing and lubricating composition is too small, the formed network may be overwhelmed by the large liquid portion making the solids unable to support the liquids in the network, which can then result in substantial migration by the liquid portions into the matrix of the fabric of the product.

In order to avoid this potential problem of having too high of a proportion of liquids, the moisturizing and lubricating compositions of the present invention comprise no more than about 50% components that are liquid at room temperature, and no less than about 50% of components that are solid at room temperature. Moisturizing and lubricating compositions that are comprised of such components provide a favorable balance of liquid and solid components, and allow for easy processability while maintaining good aesthetic attributes.

As noted throughout herein, compatibility is important in providing a moisturizing and lubricating composition that is easily processable and stable. As such, the moisturizing and lubricating compositions described herein possess high compatibility and have at least about 85% (by weight), desirably at least about 90% (by weight) more desirably at least about 94% (by weight), and even more desirably at least about 97% (by weight) of the components in a single phase at a temperature of from about 45° C. to about 80° C. With such high compatibility, the moisturizing and lubrication compositions provide a significant advantage over prior compositions and are easily processable and stable. Additionally, it is preferable that the freezing point of the compositions be higher than room temperature to avoid the need for a cooling mechanism to process the compositions.

In addition to the emollient, humectant, immobilizing agent, and compatibilizing components described herein, the moisturizing and lubricating compositions of the present invention may optionally include a skin barrier enhancing agent, such as a fat or oil (triglyceride/essential and non-essential fatty acid containing), to enhance the barrier function of the stratum corneum layer of the skin or mucous membrane. Skin and mucous membranes are comprised mainly of cholesterol, ceramides, and fatty acids. The addition of fats, oils, or triglycerides alone or in combination with sterols or sterol derivatives to the moisturizing and lubricating composition can refat/replenish or enhance the natural lipid barrier of the skin or mucous membranes that comes in contact with the cover of the absorbent product comprising the moisturizing and lubricating composition. In addition to reforming structures needed to provide the proper lipid structures in the skin, the natural fats and oils will not substantially negatively affect the natural skin barrier if repair is not necessary.

The skin barrier enhancing agent is typically from about 0.1% (by weight) to about 30% (by weight), more preferably from about 0.5% (by weight) to about 20% (by weight), and still more preferably from about 1% (by weight) to about 10% (by weight). Suitable fats or oils, or mixtures thereof, for inclusion in the moisturizing and lubricating compositions of the present invention typically comprise glycerides, triglycerides and/or essential and/or non-essential fatty acids. Suitable examples include apricot kernel oil, avocado oil, babassu oil, borage seed oil, butter, C₁₂-C₁₈ acid triglyceride, camellia oil, canola oil, caprylic/capric/lauric triglyceride, caprylic/capric/linoleic triglyceride, caprylic/capric/stearic triglyceride, caprylic/capric triglyceride, carrot oil, cashew nut oil, castor oil, cherry pit oil, chia oil, cocoa butter, coconut oil, cod liver oil, corn germ oil, corn oil, cottonseed oil, C₁₀-C₁₈ triglycerides, egg oil, epoxidized soybean oil, evening primrose oil, glyceryl triacetyl hydroxystearate, glyceryl triacetyl ricinoleate, glycosphingolipids, grape seed oil, hazelnut oil, human placental lipids, hybrid safflower oil, hybrid sunflower seed oil, hydrogenated castor oil, hydrogenated castor oil laurate, hydrogenated coconut oil, hydrogenated cottonseed oil, hydrogenated C₁₂-C₁₈ triglycerides, hydrogenated fish oil, hydrogenated lard, hydrogenated menhaden oil, hydrogenated mink oil, hydrogenated orange roughy oil, hydrogenated palm kernel oil, hydrogenated palm oil, hydrogenated peanut oil, hydrogenated shark liver oil, hydrogenated soybean oil, hydrogenated tallow, hydrogenated vegetable oil, lard, lauric/palmitic/oleic triglyceride, lesquerella oil, linseed oil, macadamia nut oil, maleated soybean oil, meadowfoam seed oil, menhaden oil, mink oil, moringa oil, mortierella oil, neatsfoot oil, oleic/linoleic triglyceride, oleic/palmitic/lauric/myristic/linoleic triglyceride, oleostearine, olive husk oil, olive oil, omental lipids, orange roughy oil, palm kernel oil, palm oil, peach kernet oil, peanut oil, pengawar djambi oil, pentadesma butter, phospholipids, pistachio nut oil, placental lipids, rapeseed oil, rice bran oil, safflower oil, sesame oil, shark liver oil, shea butter, soybean oil, sphingolipids, sunflower seed oil, sweet almond oil, tall oil, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, vegetable oil, walnut oil, wheat bran lipids, wheat germ oil, and zadoary oil.

When the moisturizing and lubricating compositions of the present invention comprise a skin enhancing agent such as a fat or oil described above, it is highly preferred that the composition also comprise an antioxidant. Compositions suitable for use on absorbent and tissue products that contain a fat or oil skin enhancing agent without an antioxidant tend to develop an offensive odor making the product commercially unsuitable. This problem is especially acute when the products are exposed to elevated temperatures during processing, storage and shipping. It has been discovered that the offensive odor is attributable to the partial or complete oxidation of the fat or oil. As such, by introducing an antioxidant into the moisturizing and lubricating compositions of the present-invention, the development of an offensive odor over time can be substantially minimized or eliminated resulting in a substantially improved commercial product.

In addition to minimizing or eliminating offensive odors which can be produced by natural fats or oils comprising the moisturizing and lubricating compositions of the present invention, the antioxidant may provide a skin health benefit by repairing damaged lipids on the skin's surface. The epidermal lipids consist of a high proportion of polyunsaturated fatty acids, which are susceptible to oxidation on the skin's surface. Oxidation of these polyunsaturated fatty acids can be initiated by a number of means including metals, such as iron, which are naturally present in the skin as well as in menstrual fluid. Antioxidants can prevent or repair oxidative damage to polyunsaturated fatty acids as well as other oxidation prone constituents in the skin.

The antioxidant is present in the moisturizing and lubricating compositions of the present invention in an amount of from about 0.05% (by weight) to about 5% (by weight), preferably from about 0.1% (by weight) to about 2% (by weight), and more preferably from about 0.1% (by weight) to about 1% (by weight). Antioxidants suitable for reducing the likelihood of the fats and/or oils to oxidize and produce an unwanted odor include natural and synthetic tocopherol, butylated hydroxyanisole (BHA), butylated hydroxytoluene (BHT), carotenoids, filtered wheat germ oil, gamma oryzanol, sodium sulfite, grape seed extract, green tea extract, rosmaric acid, ubiquinone, lipoic acid, N-acetyl-cysteine, avocado, sage, and proanthrocyanidins. Particularly preferred antioxidants include natural and synthetic tocopherol, BHT, and gamma oryzanol. Synthetic tocopherols include, for example tocopherol acetate, tocopherol linoleate, tocopherol succinate, tocopherol sorbate, tocotrienol, and Trolox (6-hydroxy-2, 5, 7, 8-tetramethyl chromane-2carboxylic acid).

The moisturizing and lubricating compositions of the present invention may also optionally include a sterol or sterol derivative or mixture of sterols and sterol derivatives to provide a skin health benefit. Typically, the moisturizing and lubricating compositions may include from about 0.1% (by weight) to about 10% (by weight), preferably from about 0.5% (by weight) to about 5% (by weight) and more preferably about 1% (by weight) of sterol or sterol derivative. Suitable sterols and sterol derivatives for incorporation into the moisturizing and lubricating compositions of the present invention include, for example, cholesterol sulfate, beta-sterols having a tail on the 17 position and having no polar groups, for example cholesterol, sitosterol, stigmasterol, and ergosterol, as well as C₁₀-C₃₀ cholesterol/lanosterol esters, cholecalciferol, cholesteryl hydroxystearate, cholesteryl isostearate, cholesteryl stearate, 7-dihydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, cholesterol esters, sterol esters, and the like, as well as mixtures thereof.

The moisturizing and lubricating compositions of the present invention may also optionally include other components such as emulsifiers, surfactants, water, viscosity modifiers, pH modifiers, enzyme inhibitors/inactivators, suspending agents, pigments, dyes, ceramides such as glucosylceramides, colorants, buffers, perfumes, antibacterial actives, antifungal actives, pharmaceutical actives, film formers, deodorants, opacifiers, astringents, solvents, organic acids, coloring agents, preservatives, antivirul actives, drugs, vitamins, aloe vera, panthenol, natural moisturizing factors, and the like. These materials are known in the art and are used in their art-established manner at their art-established amounts.

The moisturizing and lubricating compositions of the present invention are introduced onto the desired product in an amount sufficient to provide a moisturizing and lubricating benefit. For example, the moisturizing and lubricating compositions of the present invention may be introduced onto the bodyfacing surface of an absorbent product or one or both faces of a tissue product in an amount of from about 0.05 g/m² to about 100 g/m², more preferably from about 1.0 g/m² to about 40 g/m², and even more preferably from about 4 g/m² to about 15 g/m².

The present invention is illustrated by the following Examples, which are not meant to be limiting in any manner.

### EXAMPLE 1

In this Example, five moisturizing and lubricating formulations of the present invention were prepared and evaluated for various properties including water vapor transmission rate, static hygroscopicity, and stability. The components of each of the five formulations of the present invention, designated AB, AC, AG, AL, and AK are set forth in Tables 1-5 below:

TABLE 1
(AB):

| Component | Weight Percent |
|---|---|
| Glycerin | 10.0 |
| PEG (600) | 15.0 |
| PEG (1000) | 45.0 |
| PEG (10,000) | 25.0 |
| Dow Corning 200 Fluid, 100 | 2.5 |
| cst. | |
| Dow Corning 1503 Fluid | 2.5 |

TABLE 2
(AC):

| Component | Weight Percent |
|---|---|
| Glycerin | 10.0 |
| PEG 600 | 20.0 |
| PEG 1000 | 45.0 |
| Stearic Acid | 20.0 |
| Dow Corning 200 Fluid, 100 | 2.5 |
| cst. | |
| Dow Corning 1503 Fluid | 2.5 |

TABLE 3
(AG):

| Component | Weight Percent |
|---|---|
| Glycerin | 10.0 |
| PEG 600 | 20.0 |
| PEG 1000 | 30.0 |
| Stearic Acid | 20.0 |
| Dow Corning 200 Fluid, 100 | 2.5 |
| cst. | |
| Dow Corning 5329 | 15.0 |
| Dow Corning 1503 Fluid | 2.5 |

TABLE 4
(AK):

| Component | Weight Percent |
|---|---|
| PEG 600 | 15.0 |
| PEG 1000 | 20.0 |
| PEG 3350 | 25.0 |
| PEG 10,000 | 25.0 |
| Dow Corning 200 Fluid, 100 | 2.5 |
| cst. | |
| Dow Corning 1503 Fluid | 2.5 |
| Glycerin | 10.0 |

TABLE 5
(AL):

| Component | Weight Percent |
|---|---|
| Glycerin | 10.0 |
| PEG 600 | 15.0 |
| PEG 1000 | 45.0 |
| PEG 10,000 | 20.0 |
| Stearic Acid | 5.0 |
| Dow Corning 200 Fluid, 100 | 2.5 |
| cst. | |
| Dow Corning 1503 Fluid | 2.5 |

The water vapor transmission rate of the various formulations was measured to evaluate the barrier properties of the various formulations toward the permeation of water vapor. The formulations were measured by melting each formulation and manually applying the melted formulation to a collagen film using a finger cot at a level of about 2 microliters/cm². The coated collagen film was mounted onto a stainless steel permeability cup partially filled with deionized water. A gasket and a washer were placed on top of the collagen film and secured in place using metal clamps. The entire assemblage was then weighed at time zero, and then successively after 1, 2, 3, and 4 hours. The data was used to determine water vapor transmission rate through both coated and uncoated (control) films. The water vapor transmission rate was calculated from the slope of the cumulative weight loss versus time and expressed as weight loss per unit area per unit time (g/m²/h). All measurements were taken in a controlled environment (24° C. and 45% relative humidity). Each formulation was run in five replicates and the average water vapor transmission rate was calculated.

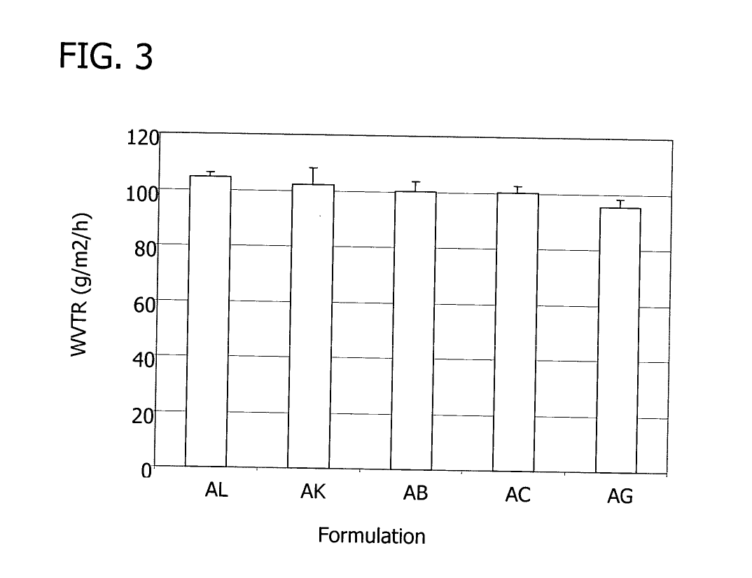

The results of the water vapor transmission tests are shown in FIG. 3. The data show that all of the formulations are generally highly permeable to water vapor, which means that each formulation will allow water to pass through it relatively easily.

Static hygroscopicity measurements were made to evaluate the ability of the formulations to absorb and hold water by quantifying the amount of water picked up from a humidified atmosphere. Each formulation was melted and applied to a preheated glass slide of known weight. Another heated glass slide was used to spread the formulation across the first slide and then the first slide weighted again to determine the amount of formulation applied. The slide was then placed for 24 hours on a rack within a humidification chamber (10% glycerin in water, relative humidity about 100%). At the conclusion of the incubation period, the slide was removed and immediately weighed. The amount of water absorbed was expressed as percentage weight of the original formulation (percent increase in sample weight). An uncoated glass slide was used as the control. Each formulation was run in five replicates and the average was calculated.

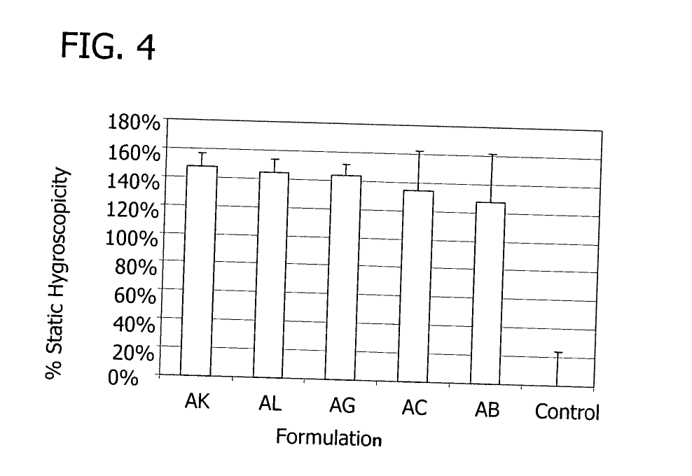

The results of the static hygroscopicity tests are shown in FIG. 4. All formulations tested showed high hygroscopicity as evidenced by more than doubling their weight in the humidified chamber.

The stability of each formulation was measured to determine what percentage of the formulation remained in a single homogeneous phase over a period of time at an elevated temperature. Each formulation was prepared, heated to melt the formulation, introduced into a 150 mL separation funnel, and stored overnight at 70° C. to determine how much formulation remained in a single phase. The following day, any phases present in the separation funnel were separated into different beakers and weighed. It was found that all of the formulations had at least about 95% (by weight) in a single phase with the exception of formulation AG, which had at least about 97% (by weight) in a single phase.

Without being bound to a particular theory, it is believed that the silicones present in the formulations described herein are the only components that may have a tendency to quickly separate out from the bulk of the formulation. Although this may not occur with every formulation, it has been discovered that the addition of a dispersing agent, such as Dow Corning 5329, can substantially reduce the amount of silicones that separate out from the formulation. As such, it is believed that formulation AG had a higher amount of components (by weight) in a single phase due to the presence of a dispersing agent in the formulation.

In view of the above, it will be seen that the several objects of the invention are achieved. As various changes could be made in the above-described products and methods without departing from the scope of the invention, it is intended that all matter contained in the above description be interpreted as illustrative and not in a limiting sense.

1. A moisturizing and lubricating composition comprising from about 1% (by weight) to about 40% (by weight) of an emollient, from about 1% (by weight) to about 20% (by weight) of a humectant, from about 30% (by weight) to about 90% (by weight) an immobilizing agent, and from about 1% (by weight) to about 40% (by weight) of a compatibilizing agent wherein no more than about 50% (by weight) of the components are liquid at room temperature and no less than about 50% of the components are solid at room temperature, and wherein at least about 85% (by weight) of the components of the moisturizing and lubricating composition form a single phase at a temperature of from about 45° C. to about 80° C.

2. The moisturizing and lubricating composition as set forth in claim 1 wherein the emollient is selected from the group consisting of petroleum based emollients, fatty acids, fatty acid esters, vegetable oils, hydrogenated vegetable oils, alkyl ethoxylates, fatty alcohols, silicones, and combinations thereof.

3. The moisturizing and lubricating composition as set forth in claim 2 wherein the silicones are selected from the group consisting of dimethicone, dimethoconol, PEG dimethicone, alkyl silicones, phenyl silicones, silicone phospholipids, and combinations thereof.

4. The moisturizing and lubricating composition as set forth in claim 1 wherein the humectant is present in an amount of from about 5% (by weight) to about 15% (by weight).

5. The moisturizing and lubricating composition as set forth in claim 1 wherein the humectant is selected from the group consisting of N-Acetyl ethanolamine, urocanic acid, aloe vera gel, arginine PCA, chitosan PCA, copper PCA, corn glycerides, dimethyl imidazolidinone, fructose, glucamine, glucose, glucose glutamate, glucuronic acid, glutamic acid, glycereth-7, glycereth-12, glycereth-20, glycereth-26, glycerin, honey, hydrogenated honey, hydrogenated starch hydrolysates, hydrolyzed corn starch, lactamide MEA, lactic acid, lactose lysine PCA, mannitol, methyl gluceth-10, methyl gluceth-20, PCA, PEG-2 lactamide, PEG-10 propylene glycol, polyamino sugar condensate, potassium PCA, propylene glycol, propylene glycol citrate, polyamino acid, polysaccharide, saccharide hydrolysate, saccharide isomerate, sodium aspartate, sodium lactate, sodium PCA, sorbitol, TEA-lactate, TEA-PCA, Urea, Xylitol, and mixtures thereof.

6. The moisturizing and lubricating composition as set forth in claim 1 wherein the humectant is selected from the group consisting of polyols, glycerine, ethoxylated glycerine, polyethylene glycols, hydrogenated starch hydrolsates, propylene glycol, silicone glycol, pyrrolidone carboxylic acid, and mixtures thereof.

7. The moisturizing and lubricating composition as set forth in claim 1 wherein the humectant is glycerin.

8. The moisturizing and lubricating composition as set forth in claim 1 wherein the immobilizing agent is present in an amount of from about 40% (by weight) to about 70% (by weight).

9. The moisturizing and lubricating composition as set forth in claim 1 wherein the immobilizing agent is selected from the group consisting of aluminum stearate, calcium stearate, magnesium stearate, zinc stearate, C₁₄-C₂₂ fatty alcohols, C₁₂-C₂₂ fatty acids, solid fatty acid esters, C₁₂-C₂₂ fatty alcohol ethoxylates having an average degree of ethoxylation ranging from about 2 to about 30, and high molecular weight polyethylene glycols having the formula:
H(OCH₂CH₂)ₓOH
wherein x is the degree of ethoxylation and is an average value of at least about 20 moles.

10. The moisturizing and lubricating composition as set forth in claim 9 wherein the polyethylene glycols are selected from the group consisting of PEG 1000, PEG 3350, PEG 6000, PEG 8000, and PEG 10,000.

11. The moisturizing and lubricating composition as set forth in claim 9 wherein the fatty alcohol is selected from the group consisting of cetyl alcohol, cetearyl alcohol, stearyl alcohol, behenyl alcohol, and mixtures thereof.

12. The moisturizing and lubricating composition as set forth in claim 1 wherein the compatibilizing agent is selected from the group consisting of propylene glycol, butylene glycol, 1,3 butylene glycol, low molecular weight polyethylene glycols (molecular weights of less than about 720 and liquid at room temperature), methoxyisopropanol, dipropylene glycol propyl ether, dipropylene glycol butyl ether, dipropylene glycol, methyl propanediol, and soluble/dispersible polypropylene glycols, and combinations thereof.

13. The moisturizing and lubricating composition as set forth in claim 1 further comprising a dispersing agent.

14. The moisturizing and lubricating composition as set forth in claim 13 wherein the dispersing agent is selected from the group consisting of polyether ethoxylated/propoxylated modified polydimethylsiloxanes which are fully or partially compatible with polydimethylsiloxanes, silicone polyethers having at least 30% siloxane, between 10% and 40% ethoxylation and between 0% and 40% propoxylation and combinations thereof.

15. The moisturizing and lubricating composition as set forth in claim 1 further comprising a skin barrier enhancing agent in an amount of from about 0.1% (by weight) to about 30% (by weight).

16. The moisturizing and lubricating composition as set forth in claim 15 wherein the skin barrier enhancing agent is selected from the group consisting of a fat and an oil.

17. The moisturizing and lubricating composition as set forth in claim 15 wherein the skin barrier enhancing agent is selected from the group consisting of apricot kernel oil, avocado oil, babassu oil, borage seed oil, butter, C₁₂-C₁₈ acid triglyceride, camellia oil, canola oil, caprylic/capric/lauric triglyceride, caprylic/capric/linoleic triglyceride, caprylic/capric/stearic triglyceride, caprylic/capric triglyceride, carrot oil, cashew nut oil, castor oil, cherry pit oil, chia oil, cocoa butter, coconut oil, cod liver oil, corn germ oil, corn oil, cottonseed oil, C₁₀-C₁₈ triglycerides, egg oil, epoxidized soybean oil, evening primrose oil, glyceryl triacetyl hydroxystearate, glyceryl triacetyl ricinoleate, glycosphingolipids, grape seed oil, hazelnut oil, human placental lipids, hybrid safflower oil, hybrid sunflower seed oil, hydrogenated castor oil, hydrogenated castor oil laurate, hydrogenated coconut oil, hydrogenated cottonseed oil, hydrogenated C₁₂-C₁₈ triglycerides, hydrogenated fish oil, hydrogenated lard, hydrogenated menhaden oil, hydrogenated mink oil, hydrogenated orange roughy oil, hydrogenated palm kernel oil, hydrogenated palm oil, hydrogenated peanut oil, hydrogenated shark liver oil, hydrogenated soybean oil, hydrogenated tallow, hydrogenated vegetable oil, lard, lauric/palmitic/oleic triglyceride, lesquerella oil, linseed oil, macadamia nut oil, maleated soybean oil, meadowfoam seed oil, menhaden oil, mink oil, moringa oil, mortierella oil, neatsfoot oil, oleic/linoleic triglyceride, oleic/palmitic/lauric/myristic/linoleic triglyceride, oleostearine, olive husk oil, olive oil, omental lipids, orange roughy oil, palm kernel oil, palm oil, peach kernet oil, peanut oil, pengawar djambi oil, pentadesma butter, phospholipids, pistachio nut oil, placental lipids, rapeseed oil, rice bran oil, safflower oil, sesame oil, shark liver oil, shea butter, soybean oil, sphingolipids, sunflower seed oil, sweet almond oil, tall oil, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, vegetable oil, walnut oil, wheat bran lipids, wheat germ oil, zadoary oil, and mixtures thereof.

18. The moisturizing and lubricating composition as set forth in claim 15 further comprising an antioxidant in an amount of from about 0.05% to about 5%.

19. The moisturizing and lubricating composition as set forth in claim 18 wherein the antioxidant is selected from the group consisting of natural and synthetic tocopherol, butylated hydroxyanisole (BHA), butylated hydroxytoluene (BHT), carotenoids, filtered wheat germ oil, gamma oryzanol, sodium sulfite, grape seed extract, green tea extract, rosmaric acid, ubiquinone, lipoic acid, N-acetyl-cysteine, avocado, sage, proanthrocyanidins, and mixtures thereof.

20. The moisturizing and lubricating composition as set forth in claim 18 wherein the antioxidant is selected from the group consisting of natural and synthetic tocopherol, butylated hydroxytoluene, gamma oryzanol, and mixtures thereof.

21. The moisturizing and lubricating composition as set forth in claim 1 further comprising a sterol or sterol derivative in an amount of from about 0.1% (by weight) to about 10% (by weight).

22. The moisturizing and lubricating composition as set forth in claim 21 wherein the sterol or sterol derivative is selected from the group consisting of cholesterol sulfate, cholesterol, sitosterol, stigmasterol, ergosterol, C₁₀-C₃₀ cholesterol/lanosterol esters, cholecalciferol, cholesteryl hydroxystearate, cholesteryl isostearate, cholesteryl stearate, 7-dihydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, cholesterol esters, sterol esters, and mixtures thereof.

23. The moisturizing and lubricating composition as set forth in claim 1 further comprising a ceramide.

24. The moisturizing and lubricating composition as set forth in claim 23 wherein the ceramide is glucosylceramide.

25. The moisturizing and lubricating composition as set forth in claim 1 further comprising one or more components selected from the group consisting of emulsifiers, surfactants, water, viscosity modifiers, pH modifiers, enzyme inhibitors/inactivators, suspending agents, pigments, dyes, colorants, buffers, perfumes, antibacterial actives, antifungal actives, natural moisturizing factors, pharmaceutical actives, film formers, deodorants, opacifiers, astringents, solvents, organic acids, preservatives, antivirul actives, drugs, vitamins, aloe vera, and panthenol.

26. The moisturizing and lubricating composition as set forth in claim 1 wherein the composition has a melting point of from about 55° C. to about 75° C.

27. The moisturizing and lubricating composition as set forth in claim 1 wherein the composition has a penetration hardness of from about 1 millimeter to about 20 millimeters.

28. The moisturizing and lubricating composition as set forth in claim 1 wherein at least about 90% (by weight) of the components of the moisturizing and lubricating composition form a single phase at a temperature of from about 45° C. to about 80° C.

29. The moisturizing and lubricating composition as set forth in claim 1 wherein at least about 94% (by weight) of the components of the moisturizing and lubricating composition form a single phase at a temperature of from about 45° C. to about 80° C.

30. The moisturizing and lubricating composition as set forth in claim 1 wherein at least about 97% (by weight) of the components of the moisturizing and lubricating composition form a single phase at a temperature of from about 45° C. to about 80° C.

31. A moisturizing and lubricating composition comprising from about 1% (by weight) to about 40% (by weight) of a silicone, from about 1% (by weight) to about 20% (by weight) of a humectant, from about 30% (by weight) to about 90% (by weight) an immobilizing agent, from about 1% (by weight) to about 40% (by weight) of a compatibilizing agent and a dispersing agent wherein no more than about 50% (by weight) of the components are liquid at room temperature and no less than about 50% of the components are solid at room temperature, and wherein at least 85% (by weight) of the components of the moisturizing and lubricating composition form a single phase at a temperature of from about 45° C. to about 80° C.

32. The moisturizing and lubricating composition as set forth in claim 31 wherein the weight ratio of silicone to dispersing agent is at least about 1:1.

33. The moisturizing and lubricating composition as set forth in claim 31 wherein the dispersing agent is Dow Corning 5329.

34. The moisturizing and lubricating composition as set forth in claim 31 wherein the silicone is selected from the group consisting of dimethicone, dimethoconol, PEG dimethicone, alkyl silicones, phenyl silicones, silicone phospholipids, and combinations thereof.

35. The moisturizing and lubricating composition as set forth in claim 31 wherein the humectant is present in an amount of from about 5% (by weight) to about 15% (by weight).

36. The moisturizing and lubricating composition as set forth in claim 31 wherein the humectant is selected from the group consisting of N-Acetyl ethanolamine, urocanic acid, aloe vera gel, arginine PCA, chitosan PCA, copper PCA, corn glycerides, dimethyl imidazolidinone, fructose, glucamine, glucose, glucose glutamate, glucuronic acid, glutamic acid, glycereth-7, glycereth-12, glycereth-20, glycereth-26, glycerin, honey, hydrogenated honey, hydrogenated starch hydrolysates, hydrolyzed corn starch, lactamide MEA, lactic acid, lactose lysine PCA, mannitol, methyl gluceth-10, methyl gluceth-20, PCA, PEG-2 lactamide, PEG-10 propylene glycol, polyamino sugar condensate, potassium PCA, propylene glycol, propylene glycol citrate, polyamino acid, polysaccharide, saccharide hydrolysate, saccharide isomerate, sodium aspartate, sodium lactate, sodium PCA, sorbitol, TEA-lactate, TEA-PCA, Urea, Xylitol, and mixtures thereof.

37. The moisturizing and lubricating composition as set forth in claim 31 wherein the humectant is selected from the group consisting of polyols, glycerine, ethoxylated glycerine, polyethylene glycols, hydrogenated starch hydrolsates, propylene glycol, silicone glycol, pyrrolidone carboxylic acid, and mixtures thereof.

38. The moisturizing and lubricating composition as set forth in claim 31 wherein the humectant is glycerin.

39. The moisturizing and lubricating composition as set forth in claim 31 wherein the immobilizing agent is present in an amount of from about 40% (by weight) to about 70% (by weight).

40. The moisturizing and lubricating composition as set forth in claim 31 wherein the immobilizing agent is selected from the group consisting of aluminum stearate, calcium stearate, magnesium stearate, zinc stearate, C₁₄-C₂₂ fatty alcohols, C₁₂-C₂₂ fatty acids, solid fatty acid esters, C₁₂-C₂₂ fatty alcohol ethoxylates having an average degree of ethoxylation ranging from about 2 to about 30, and high molecular weight polyethylene glycols having the formula:
H(OCH₂CH₂)ₓOH
wherein x is the degree of ethoxylation and is an average value of at least about 20 moles.

41. The moisturizing and lubricating composition as set forth in claim 40 wherein the polyethylene glycols are selected from the group consisting of PEG 1000, PEG 3350, PEG 6000, PEG 8000, PEG 10,000.

42. The moisturizing and lubricating composition as set forth in claim 40 wherein the fatty alcohol is selected from the group consisting of cetearyl alcohol, cetyl alcohol, stearyl alcohol, behenyl alcohol, and mixtures thereof.

43. The moisturizing and lubricating composition as set forth in claim 31 wherein the compatibilizing agent is selected from the group consisting of propylene glycol, butylene glycol, 1,3 butylene glycol, low molecular weight polyethylene glycols (molecular weights of less than about 720 and liquid at room temperature), methoxyisopropanol, dipropylene glycol propyl ether, dipropylene glycol butyl ether, dipropylene glycol, methyl propanediol, and soluble/dispersible polypropylene glycols.

44. The moisturizing and lubricating composition as set forth in claim 31 further comprising a skin barrier enhancing agent in an amount of from about 0.1% (by weight) to about 30% (by weight).

45. The moisturizing and lubricating composition as set forth in claim 44 wherein the skin barrier enhancing agent is selected from the group consisting of a fat and an oil.

46. The moisturizing and lubricating composition as set forth in claim 44 wherein the skin barrier enhancing agent is selected from the group consisting of apricot kernel oil, avocado oil, babassu oil, borage seed oil, butter, C₁₂-C₁₈ acid triglyceride, camellia oil, canola oil, caprylic/capric/lauric triglyceride, caprylic/capric/linoleic triglyceride, caprylic/capric/stearic triglyceride, caprylic/capric triglyceride, carrot oil, cashew nut oil, castor oil, cherry pit oil, chia oil, cocoa butter, coconut oil, cod liver oil, corn germ oil, corn oil, cottonseed oil, C₁₀-C₁₈ triglycerides, egg oil, epoxidized soybean oil, evening primrose oil, glyceryl triacetyl hydroxystearate, glyceryl triacetyl ricinoleate, glycosphingolipids., grape seed oil, hazelnut oil, human placental lipids, hybrid safflower oil, hybrid sunflower seed oil, hydrogenated castor oil, hydrogenated castor oil laurate, hydrogenated coconut oil, hydrogenated cottonseed oil, hydrogenated C₁₂-C₁₈ triglycerides, hydrogenated fish oil, hydrogenated lard, hydrogenated menhaden oil, hydrogenated mink oil, hydrogenated orange roughy oil, hydrogenated palm kernel oil, hydrogenated palm oil, hydrogenated peanut oil, hydrogenated shark liver oil, hydrogenated soybean oil, hydrogenated tallow, hydrogenated vegetable oil, lard, lauric/palmitic/oleic triglyceride, lesquerella oil, linseed oil, macadamia nut oil, maleated soybean oil, meadowfoam seed oil, menhaden oil, mink oil, moringa oil, mortierella oil, neatsfoot oil, oleic/linoleic triglyceride, oleic/palmitic/lauric/myristic/linoleic triglyceride, oleostearine, olive husk oil, olive oil, omental lipids, orange roughy oil, palm kernel oil, palm oil, peach kernet oil, peanut oil, pengawar djambi oil, pentadesma butter, phospholipids, pistachio nut oil, placental lipids, rapeseed oil, rice bran oil, safflower oil, sesame oil, shark liver oil, shea butter, soybean oil, sphingolipids, sunflower seed oil, sweet almond oil, tall oil, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, vegetable oil, walnut oil, wheat bran lipids, wheat germ oil, zadoary oil, and mixtures thereof.

47. The moisturizing and lubricating composition as set forth in claim 44 further comprising an antioxidant in an amount of from about 0.05% to about 5%.

48. The moisturizing and lubricating composition as set forth in claim 47 wherein the antioxidant is selected from the group consisting of natural and synthetic tocopherol, butylated hydroxyanisole (BHA), butylated hydroxytoluene (BHT), carotenoids, filtered wheat germ oil, gamma oryzanol, sodium sulfite, grape seed extract, green tea extract, rosmaric acid, ubiquinone, lipoic acid, N-acetyl-cysteine, avocado, sage, proanthrocyanidins, and mixtures thereof.

49. The moisturizing and lubricating composition as set forth in claim 47 wherein the antioxidant is selected from the group consisting of natural and synthetic tocopherol, butylated hydroxytoluene, gamma oryzanol, and mixtures thereof.

50. The moisturizing and lubricating composition as set forth in claim 31 further comprising a sterol or sterol derivative in an amount of from about 0.1% (by weight) to about 10% (by weight).

51. The moisturizing and lubricating composition as set forth in claim 50 wherein the sterol or sterol derivative is selected from the group consisting of cholesterol sulfate, cholesterol, sitosterol, stigmasterol, ergosterol, C₁₀-C₃₀ cholesterol/lanosterol esters, cholecalciferol, cholesteryl hydroxystearate, cholesteryl isostearate, cholesteryl stearate, 7-dihydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, cholesterol esters, sterol esters, and mixtures thereof.

52. The moisturizing and lubricating composition as set forth in claim 31 further comprising one or more components selected from the group consisting of emulsifiers, surfactants, water, viscosity modifiers, natural moisturizing factors, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, antivirul actives, drugs, vitamins, aloe vera, and panthenol.

53. The moisturizing and lubricating composition as set forth in claim 31 wherein the moisturizing and lubricating composition has a melting point of from about 55° C. to about 75° C.

54. The moisturizing and lubricating composition as set forth in claim 31 wherein the composition has a penetration hardness of from about 1 millimeter to about 20 millimeters.

55. The moisturizing and lubricating composition as set forth in claim 31 further comprising a ceramide.

56. The moisturizing and lubricating composition as set forth in claim 55 wherein the ceramide is glucosylceramide.

57. The moisturizing and lubricating composition as set forth in claim 31 wherein at least about 90% (by weight) of the components of the moisturizing and lubricating composition form a single phase at a temperature of from about 45° C. to about 80° C.

58. The moisturizing and lubricating composition as set forth in claim 31 wherein at least about 94% (by weight) of the components of the moisturizing and lubricating composition form a single phase at a temperature of from about 45° C. to about 80° C.

59. The moisturizing and lubricating composition as set forth in claim 31 wherein at least about 97% (by weight) of the components of the moisturizing and lubricating composition form a single phase at a temperature of from about 45° C. to about 80° C.
